(19) United States
(12) Patent Application Publication
(10) Pub. No.: US 2003/0186915 A1
(43) Pub. Date: Oct. 2, 2003

(54) Regulatory polynucleotides and uses thereof

(76) Inventors: Yang Pan, Bellevue, WA (US); Bradley R. Sevetson, Seattle, WA (US); Jonathan M.J. Derry, Bainbridge Island, WA (US)

Correspondence Address: IMMUNEX CORPORATION, LAW DEPARTMENT, 51 UNIVERSITY STREET, SEATTLE, WA, 98101

(21) Appl. No.: 10/365,737

(22) Filed: Feb. 11, 2003

Related U.S. Application Data
(60) Provisional application No. 60/356,212, filed on Feb. 11, 2002.

(51) Int. Cl. A61K 48/00; A61K 38/29; A61K 31/663; C12Q 1/68; C07H 21/04; C12P 21/02; C12N 5/06; C12N 9/64

(52) U.S. Cl. 514/44; 514/12; 514/102; 435/6; 435/69.1; 435/226; 435/320.1; 435/325; 536/23.2

(57) ABSTRACT

The present invention provides regulatory polynucleotides, vectors, and cells containing these polynucleotides. More particularly, this invention relates to regulatory polynucleotides derived from a regulatory region of the SOST gene, and the use of such polynucleotides for screening for agents that affect SOST regulation, for tissue-specific gene expression, and for other therapeutic and diagnostic applications. This invention further relates to methods of modulating bone mass in humans and other animals and for the treatment of osteoporosis and related bone disorders.

### CROSS-REFERENCE TO RELATED APPLICATIONS

This application hereby claims the benefit of U.S. provisional application serial No. 60/356,212, filed Feb. 11, 2002, the entire disclosure of which is relied upon and incorporated by reference.

### FIELD OF THE INVENTION

This invention relates to regulatory polynucleotides, vectors, and cells containing these polynucleotides, and the use of these polynucleotides for tissue specific gene expression, screening for agents that affect SOST regulation, as well as methods of modulating bone mass in humans and other animals, and for the treatment of osteoporosis and related metabolic disorders.

### BACKGROUND

Degenerative bone diseases and disorders are a source of morbidity and mortality in vertebrate organisms. The most common metabolic bone disorder is osteoporosis. Osteoporosis can generally be defined as the reduction in the quantity of bone, or the atrophy of skeletal tissue. In general, there are two types of osteoporosis: primary and secondary. “Secondary osteoporosis” is the result of an identifiable disease process or agent. Approximately 90% of all osteoporosis cases are “primary osteoporosis”. Such primary osteoporosis includes postmenopausal osteoporosis, age-associated osteoporosis (affecting a majority of humans over the age of 70 to 80), and idiopathic osteoporosis affecting middle-aged and younger men and women.

For some osteoporotic individuals the loss of bone tissue is sufficiently great so as to cause mechanical failure of the bone structure. Bone fractures often occur, for example, in the hip and spine of women suffering from postmenopausal osteoporosis. Kyphosis (abnormally increased curvature of the thoracic spine) may also result.

The mechanism of bone loss in subjects afflicted with osteoporosis is believed to involve an imbalance in the process of “bone remodeling”. Bone remodeling occurs throughout life, renewing the skeleton and maintaining the strength of bone. This remodeling involves the erosion and filling of discrete sites on the surface of bones, by an organized group of cells called “basic multicellular units” or “BMUs”. BMUs primarily consist of “osteoclasts”, “osteoblasts”, and their cellular precursors. In the remodeling cycle, bone is resorbed at the site of an “activated” BMU by an osteoclast, forming a resorption cavity. This cavity is then filled with bone by osteoblasts.

Normally, in adults, the remodeling cycle results in a small deficit in bone, due to incomplete filling of the bone resorption cavity. Thus, even in healthy adults, age-related bone loss occurs. However, in subjects afflicted with osteoporosis, there is an increase in the number of BMUs that are activated. This increased activation accelerates bone remodeling, resulting in abnormally high bone loss.

Factors involved in bone formation include hormones such as estrogen, calcitonin, and parathyroid hormone (PTH); growth factors such as bone morphogenic protein (BMP); and chemicals such as active vitamin D, calcium preparations, and vitamin K2. Estrogen, calcitonin, active vitamin D, and calcium preparations are used as medicine for controlling bone mass in osteoporosis or similar cases. In particular, high dosages of dietary calcium, with or without vitamin D, are commonly recommended to as an osteoporosis preventative to postmenopausal women. Bone morphogenic protein (BMP), also known as osteogenic protein, is a family of cytokines known to regulate cartilage and bone differentiation in vivo. BMP is thought to effectively form bone (cartilaginous ossification) by replacing cartilaginous callus with new bone cells in repairing fractures or bone deficits (Duprez et al., Dev. Biol. 174:448-452, 1996; Nakase et al., J. Bone Miner. Res. 9:651-659, 1994).

Many compositions and methods have been described in the medical literature for the treatment of osteoporosis. These compositions and methods attempt to either slow the loss of bone or to produce a net gain in bone mass. See, for example, R. C. Haynes, Jr. et al., “Agents affecting Calcification”, The Pharmacological Basis of Therapeutics, 7th Edition (A. G. Gilman, L. S. Goodman et al., Editors, 1985); G. D. Whedon et al., “An Analysis of Current Concepts and Research Interest in Osteoporosis”, Current Advances in Skeletogenesis (A. Ornoy et al., Editors, 1985); and W. A. Peck, et al., Physician's Resource Manual on Osteoporosis (1987), published by the National Osteoporosis Foundation. Among the treatments for osteoporosis suggested in the literature is the administration of bisphosphonates or other bone-active phosphonates (see, e.g., Storm et al., New Engl. J. of Med., 322:1265, 1990; and Watts et al., New Engl. J. of Med., 323:73, 1990). Such treatments using a variety of bisphosphonates are described in U.S. Pat. Nos. 4,761,406; 4,812,304; 4,812,311; and 4,822,609. The use of such phosphonates for the treatment of osteoporosis, and other disorders involving abnormal calcium and phosphate metabolism, is also described in U.S. Pat. Nos. 3,683,080; 4,330,537; and 4,267,108; European Patent Publication 298,553; and Francis et al., “Chemical, Biochemical, and Medicinal Properties of the Diphosphonates”, The Role of Phosphonates in Living Systems, Chapter 4 (1983). Parathyroid hormone has also been suggested as a therapy for osteoporosis. Such treatments using parathyroid hormone are disclosed in Hefti, et al., Clin. Sci. 62:389-396, 1982; German Patent Publication DE 39 35 738; U.S. Pat. Nos. 4,698,328; and 4,833,125.

Most currently approved therapeutic agents for osteoporosis are antiresorptives. As such, they are not effective in patients with established osteoporosis. In addition, estrogen therapy has prescribed as a preventative treatment for osteoporosis in postmenopausal women. However, concerns as to the health effects of estrogen therapy have cast doubt on that treatment. Thus, there remains a need to develop additional therapeutic agents which prevent osteoporosis, as well as treat individuals already inflicted with the condition.

### SUMMARY OF THE INVENTION

The invention provides isolated nucleic acid molecules comprising regulatory polynucleotides that are at least 80% identical to SEQ ID NO:1, or its complement, or polynucleotides that are at least 80% identical to a fragment of SEQ ID NO: 1, or its complement, and that regulate the expression of a nucleic acid molecule operably linked thereto. The regulatory polynucleotides of the present invention include polynucleotides that hybridize to SEQ ID NO: 1, its complement, or a fragment thereof, under moderate to stringent conditions, and that regulate the expression of a nucleic acid molecule operably linked thereto. These polynucleotide fragments include, for example, nucleotides 1 to 1814 of SEQ ID NO: 1, polynucleotides which are at least 80% identical to this fragment, or complementary to this fragment; nucleotides 1673 to 1814 of SEQ ID NO: 1, polynucleotides which are at least 80% identical to this fragment or complementary to this fragment; and nucleotides 1673 to 1748 of SEQ ID NO: 1, polynucleotides which are at least 80% identical to this fragment, or complementary to this fragment, and that regulate the expression of a nucleic acid molecule operably linked thereto.

The invention also provides methods for identifying agents that modulate expression from the regulatory polynucleotides of the present invention. In one embodiment, the method includes contacting a sample containing a regulatory polynucleotide operably linked to a reporter gene with the agent and determining expression of the reporter gene compared to a control, wherein a change in expression is indicative that the agent alters transcription.

The invention also provides agents that modulate expression from the regulatory polynucleotide. Such agents include polynucleotides (e.g., antisense and ribozyme molecules), polypeptides, peptides, peptidomimetics, and small molecules.

The invention further provides methods of modulating the expression of the SOST gene by modulating transcription from the regulatory polynucleotides of the present invention. In one embodiment, the invention provides a method to stimulate bone formation by blocking the production and/or activity of the SOST protein by inhibiting expression of the SOST gene using antagonists of the regulatory polynucleotides of the present invention.

The invention provides an expression vector comprising the regulatory polynucleotides of the present invention. In one embodiment, the expression vector further comprises a reporter gene and/or a multiple cloning site.

Also provided by the invention are recombinant host cells genetically engineered to contain the regulatory polynucleotides of the present invention. In one embodiment the host cell is an osteoclast, an osteoblast, a chondrocyte, a hepatocyte, or a renal cell.

The invention also provides compositions comprising a host cell, expression vector, or agent of the invention and a pharmaceutically acceptable carrier.

The invention also provides a method of modulating bone formation in a subject, by administering an agent that modulates transcription from a regulatory polynucleotide of the invention. In another embodiment, the agent inhibits bone formation.

The invention provides a method of promoting bone formation in a subject comprising administering an agent to the subject which interacts with a regulatory polynucleotide of the present invention and inhibits expression of an SOST gene product. In one embodiment, the agent is a polynucleotide such as an antisense molecule, a polypeptide, a peptide, a peptidomimetic, and a small molecule.

The invention further provides a method of treating a bone degenerative disease or disorder in a subject comprising administering an agent to the subject which interacts with a regulatory polynucleotide of the present invention and inhibits expression of an SOST gene product. In one embodiment, the bone degenerative disease or disorder is selected from the group consisting of non-union fractures; bone cavities; tumor resection; fresh fractures; cranial/facial abnormalities; spinal fusions; cancer; arthritis; osteoarthritis; and osteoporosis.

In yet another aspect of the invention a method of increasing bone mass in a subject afflicted with osteoporosis is provided. The method includes administering an agent to the subject which interacts with a regulatory polynucleotide of the invention and inhibits expression of an SOST gene product operably linked to the regulatory polynucleotide.

### BRIEF DESCRIPTION OF THE DRAWINGS

FIG. 1 shows a regulatory polynucleotide of the invention (SEQ ID NO:1). The underlined sequences identify the consensus binding sequences for various transcription factors. These are, in a 5′ to 3′ direction, a Cbfa1 binding site, AACCACA (SEQ ID NO: 2), an upstream E box, CACGTG (SEQ ID NO: 3), a C/EBP binding site, CTTGCCTCA (SEQ ID NO: 4), and a downstream E box, CACCTG (SEQ ID NO: 5).

FIG. 2 shows the restriction sites in a regulatory polynucleotide of the invention (nucleotides 1 to 1814 of SEQ ID NO: 1), and the activity of the various restriction fragments when inserted into a reporter construct as described in the Examples below.

FIG. 3 shows the effect of MyoD and Cfa1 on the activity of both the EcoRV restriction fragment in a reporter construct, and the 1.8 kb regulatory region in a reporter construct.

### DETAILED DESCRIPTION OF THE INVENTION

The present invention provides nucleic acid molecules comprising regulatory polynucleotides capable of modulating transcription of a nucleic acid molecule operably linked thereto. The present invention also provides methods for identifying agents that modulate expression from the regulatory polynucleotides of the present invention. The present invention also provides methods of modulating the expression of the SOST gene by modulating transcription from the regulatory polynucleotides of the present invention. The present invention also provides methods of tissue-specific gene expression using the regulatory polynucleotides. The present invention also provides agents that modulate expression of the regulatory polynucleotides, such as antisense molecules. In one embodiment, the invention provides a method of stimulating bone formation by blocking the transcription of the SOST gene using antagonists of the regulatory polynucleotides. The present invention also provides recombinant cells containing recombinant constructs comprising regulatory polynucleotides of the invention.

The sclerosteosis gene (SOST) was identified from genetic studies demonstrating that loss-of-function mutations in this gene caused a rare sclerosing bone dysplasia characterized by skeletal overgrowth. This disorder, termed “sclerosteosis” is a progressive disorder characterized by general skeletal overgrowth, gigantism, entrapment of cranial nerves, increased intracranial pressure due to widening of the calvarium of the skull, and increased thickness and density of both trabecular and cortical bone. The SOST gene is expressed in osteoblasts and encodes a secreted 213 amino acid polypeptide having homology to the DAN family of secreted TGF-β family antagonists. This suggests that the SOST protein acts to repress bone growth by antagonizing TGF-β or BMP function. (Balemans et al., Human Mol. Genet., 10(5):537-543, 2001; and Brunkow et al., Am. J. Hum. Genet., 68:577-589, 2001; both of which are incorporated herein by reference).

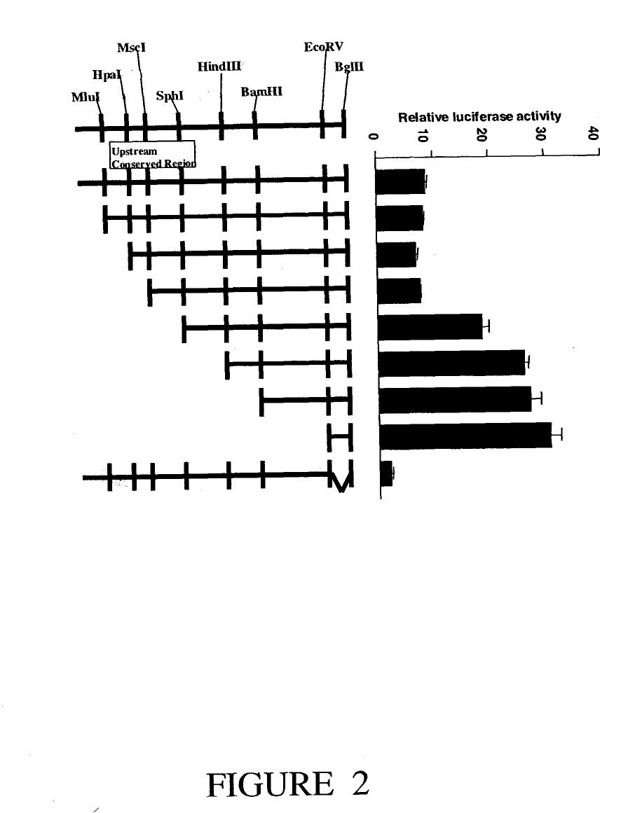

The regulatory region 5′ of the coding region of the SOST gene was identified and sequenced as described in Example 1 below. The sequence of this regulatory region is set forth in SEQ ID NO:1. Promoter activity of this regulatory region and its fragments was demonstrated in experiments using reporter constructs. The EcoRV-BglII fragment (herein referred to as the EcoRV fragment) shown in FIG. 2 was demonstrated to be particularly active as a promoter. In contrast 5′polynucleotides of the regulatory region exhibited a repressive effect on promoter activity. Agents which modify the activity of the regulatory polynucleotides such as EcoRV fragment region were also identified.

The present invention provides nucleic acid molecules comprising regulatory polynucleotides capable of modulating transcription of a nucleic acid molecule operably linked thereto. These regulatory polynucleotides include polynucleotides having sequences at least 80% identical to SEQ ID NO:1 or its complement, and that regulate the expression of a nucleic acid molecule operably linked thereto. The regulatory polynucleotides include polynucleotides that hybridizes to a polynucleotide having SEQ ID NO: 1, or its complement, or a fragment thereof, under moderate to stringent conditions, and that regulate the expression of a nucleic acid molecule operably linked thereto. The regulatory polynucleotides also include fragments of SEQ ID NO: 1 or their complements, or polynucleotides having sequences at least 80% identitical to the fragments or their complements, and that regulate the expression of a nucleic acid molecule operably linked thereto. These polynucleotide fragments include, for example, nucleotides 1 to 1814 of SEQ ID NO: 1, polynucleotides which are at least 80% identical to this fragment, or its complement, and that regulate the expression of a nucleic acid molecule operably linked thereto; nucleotides 1673 to 1814 of SEQ ID NO: 1, and polynucleotides which are at least 80% identical to this fragment, or its complement, and that regulate the expression of a nucleic acid molecule operably linked thereto; and nucleotides 1673 to 1748 of SEQ ID NO: 1, polynucleotides that are at least 80% identical to this fragment or its complement, and that regulate the expression of a nucleic acid molecule operably linked thereto. The regulatory polynucleotides of the present invention also includes polynucleotides wherein the nucleotide base can be a modified base and/or wherein the nucleotide of T can also be U.

As used herein, a “polynucleotide” refers to a polymeric form of nucleotides of at least 5 nucleotides in length. The term “polynucleotide” as used herein is used synonymously with “oligonucleotide”, which is typically 2 to 50 nucleotides in length. The nucleotides can be ribonucleotides (RNA), deoxyribonucleotides (DNA), or modified forms of either type of nucleotide and may also include related residues such as, for example, inosine (1). The term includes single and double stranded forms of DNA or RNA. DNA includes, for example, cDNA, genomic DNA, chemically synthesized DNA, DNA amplified by PCR, and combinations thereof. The polynucleotides of the invention includes those derived from human sources, as well as from non-human species.

As used herein the term “polynucleotide regulatory region” refers to the region of the gene that regulates the transcription of the gene. In general, regulatory regions include regions 5′ (e.g., upstream) of the initiation codon (e.g., ATG) and/or 3′ (e.g., downstream) of the stop codon for the particular gene. For example, a polynucleotide regulatory region can include a polynucleotide sequence that functions to control the transcription of one or more genes, located upstream of the transcription initiation site of the gene, and can contain, for example, a binding site for DNA-dependent RNA polymerase, transcription initiation sites, transcription factor binding sites, repressor and activator protein binding sites, calcium or cAMP responsive sites, promoters, enhancers, a start codon (i.e., ATG) in front of the protein-encoding gene, splicing signals for introns, maintenance of the correct reading frame of that gene to permit proper translation of the mRNA, and other sequences of nucleotides known to directly or indirectly to regulate the amount of transcription from the regulatory region. As used herein “promoter” refers to a minimal sequence sufficient to direct transcription. Promoters can be constitutive and inducible (see e.g., Bitter et al., Methods in Enzymology. 153:516-544, 1987). Additional promoter related regulatory elements are those promoter elements which are sufficient to render promoter-dependent gene expression controllable for cell-type specific, tissue-specific, or inducible by external signals or agents; such elements may be located in the 5′ or 3′ regions of the gene. The regulatory region identified in the present invention is an SOST regulatory region having SEQ ID NO: 1, and fragments thereof having regulatory activity.

As used herein the term “regulatory polynucleotides” refers to polynucleotides having at least 80% identity to SEQ ID NO: 1, or its complement, or at least 80% identity to a fragment of SEQ ID NO:1, or its complement, or polynucleotides that hybridize to SEQ ID NO: 1 or its complement, or a fragment thereof, wherein the polynucleotide has regulatory activity, that is, can regulate the expression of a nucleic acid molecule operably linked thereto.

As used herein the term “regulatory activity” refers to activities that alter the expression of a nucleic acid molecule operably linked to the regulatory polynucleotide, for example, promoter activity, or activity which represses promoter activity. These activites can be measured by determining the extent of transcription of a gene or heterologous polynucleotide that is operably linked to the regulatory polynucleotide. The regulatory activity may be measured directly by measuring the amount of RNA transcript produced, for example by Northern blot or PCR, or indirectly by measuring the product coded for by the RNA transcript, such as when a reporter gene is linked to the promoter, as described in the Examples below.

As used herein “operably linked” means that a regulatory polynucleotide and a polynucleotide of interest are situated within a construct, vector, or cell in such a way that the polypeptide encoded by the polynucleotide of interest is expressed when appropriate molecules (such as polymerases) are present. In one embodiment, a construct comprising a regulatory polynucleotide of the invention is operably linked to a polynucleotide encoding a reporter molecule. Such a construct can be transformed or transfected into a host cell thereby generating a recombinant host cell. In another embodiment, a regulatory polynucleotide of the invention is integrated into the genome of a recombinant host cell such that it is operably linked to a polynucleotide encoding a polypeptide of interest. The polynucleotide encoding the polypeptide of interest may be a polynucleotide existing in the genome of the host cell or may be a polynucleotide transformed or transfected into the host cell prior to, simultaneous with, or subsequent to transformation or transfection of the host cell with the regulatory polynucleotide of the invention.

The term “regulatory agent” or “agent” refers to a biochemical agent that acts to induce or repress the expression and transcription of a polynucleotide driven by a regulatory polynucleotide under conditions such that the regulatory agent (e.g., a polymerase, a repressor, nuclear inhibitor, or inducer) interacts with the regulatory polynucleotide to permit promotion or inhibition of the polynucleotide sequence. The term “induce” refers to an increase in transcription or expression brought about by a transcriptional inducer, relative to some basal level of transcription. The term “repress” refers to a decrease in gene transcription or expression brought about by a transcriptional repressor, relative to some basal level of transcription. A regulatory agent may be a protein, a polypeptide, a peptide, a peptidomimetic, a hormone, a polynucleotide (e.g., an antisense molecule), or a small molecule.

Reporter genes or molecules (e.g., a reporter protein) that are useful in the invention include any molecule that upon transcription provides a detectable signal or product, which product may be RNA, DNA, or protein. The detection may be accomplished by any method known to one of skill in the art. For example, detection of mRNA expression may be accomplished by using Northern blots or RT-PCR amplification techniques. Detection of protein may be accomplished by staining with antibodies specific to the protein. In one embodiment, a reporter gene is operably linked in a regulatory polynucleotide such that detection of the reporter gene product provides a measure of the transcriptional activity of the regulatory polynucleotides. Examples of reporter genes include, but are not limited to, those coding for chloramphenicol acetyl transferase (CAT), luciferase, β-galactosidase, alkaline phosphatase, antibiotic resistance genes, SV40 T antigen, human growth hormone (hGH), and the like.

Regulatory polynucleotides of the present invention include nucleotide sequence lengths that are at least 25% to 90% or more (e.g., 50%, 60%, 70%, 80% or more) of the length of SEQ ID NO:1 or its fragments and have at least 60% to 99% or more (e.g., 70%, 75%, 80%, 85%, 90%, 95%, 97.5%, 99%, 99.5% or more) sequence identity with that of SEQ ID NO:1, where sequence identity is determined by comparing the nucleotide sequences when aligned so as to maximize overlap and identity while minimizing sequence gaps.

The percent identity can be determined by visual inspection and mathematical calculation. The percent identity of polynucleotide sequences can be determined by comparing sequence information using the GAP computer program, version 6.0 described by Devereux et al. (Nucl. Acids Res. 12:387, 1984) and available from the University of Wisconsin Genetics Computer Group. Typical default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; (2) a penalty of 3.0 for each gap and an additional 0.10 penalty for each symbol in each gap; and (3) no penalty for end gaps. Other programs used by those skilled in the art of sequence comparison may also be used, such as, for example, the BLASTN program version 2.0.9, available for use via the National Library of Medicine website: www.ncbi.nlm.nih.gov/gorf/wblast2.cgi, or the UW-BLAST 2.0 algorithm. Standard default parameter settings for UW-BLAST 2.0 are described at the following Internet webpage: ncbi.nlm.nih.gov/BLAST/. In addition, the BLAST algorithm typically uses the BLOSUM62 scoring matrix, and optional parameters that may be used are as follows: (A) inclusion of a filter to mask segments of the query sequence that have low compositional complexity (as determined by the SEG program of Wootton & Federhen (Computers and Chemistry, 1993); also see Wootton and Federhen, Methods Enzymol. 266:554-71, 1996) or segments consisting of short-periodicity internal repeats (as determined by the XNU program of Clayerie & States, Computers and Chemistry, 1993), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported); preferred E-score threshold values are 0.5, or in order of increasing preference, 0.25, 0.1, 0.05, 0.01, 0.001, 0.0001, 10⁻⁵, 10⁻¹⁰, 10⁻¹⁵, 10⁻²⁰, 10⁻²⁵, 10⁻³⁰, 10⁻⁴⁰, 10⁻⁵⁰, 10⁻⁷⁵, or 10⁻¹⁰⁰.

The invention also includes regulatory polynucleotides that hybridize under moderately stringent conditions, and more typically highly stringent conditions, to a regulatory polynucleotide having a sequence as set forth in SEQ ID NO:1, its fragment, or a fragment thereof. The basic parameters affecting the choice of hybridization conditions and guidance for devising suitable conditions are set forth by Sambrook et al. (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55° C. (or other similar hybridization solutions, such as one containing about 50% formamide, with a hybridization temperature of about 42° C.), and washing conditions of about 60° C., in 0.5×SSC, 0.1% SDS. Generally, highly stringent conditions are defined as hybridization conditions as above, but with washing at approximately 68° C., 0.2×SSC, 0.1% SDS. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. The wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acid molecules of known sequences are hybridized, the hybrid length can be determined by aligning the sequences of the nucleic acid molecules and identifying the region or regions of optimal sequence complementarity. The hybridization temperature for hybrids anticipated to be less than 50 base pairs in length should be 5 to 10° C. less than the melting temperature (Tₘ) of the hybrid, where Tₘ is determined according to the following equations. For hybrids less than 18 base pairs in length, Tₘ (° C.)=2(# of A+T bases)+4(# of G+C bases). For hybrids above 18 base pairs in length, Tₘ (° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165M). Typically, each such hybridizing nucleic acid molecule has a length that is at least 15 nucleotides (or at least 18 to about 20 nucleotides, or at least 25 to about 30 nucleotides, or at least 40 nucleotides, or at least 50 nucleotides), or at least 25%, at least 50%, at least 60%, at least 70%, or at least 80% of the length of a regulatory polynucleotide of the invention to which it hybridizes, and has at least 60%, at least 70% to about 75%, at least 80% to about 85%, at least 90% to about 95%, at least 97.5%, at least 99%, or at least 99.5%) with a regulatory polynucleotide of the invention to which it hybridizes, where sequence identity is determined by comparing the sequences of the hybridizing nucleic acids when aligned so as to maximize overlap and identity while minimizing sequence gaps as described above.

The regulatory polynucleotides of the present invention can be isolated from genomic nucleic acid, either from human cells or from other species having homologous regulatory polynucleotides. The polynucleotides can be isolated using techniques well-known in the art such as cross-species hybridization, or produced by PCR-based techniques, such as those described, in the Examples below. Such polynucleotides can also be produced recombinantly in vectors, grown in prokaryotic or eukaryotic cells, and then be purified from the genomic nucleic acids, using techniques well known in the art. The regulatory polynucleotides provided herein can be made and used by those skilled in the art without undue experimentation using, for example, techniques described above and in, for example, Sambrook, Fischer and Maniatis, Molecular Cloning, a Laboratory Manual, (2nd ed.), Cold Spring Harbor Laboratory Press, New York, (1989) and F. M. Ausubel et al eds., Current Protocols in Molecular Biology, John Wiley and Sons (1994), the disclosure of which is incorporated herein by reference.

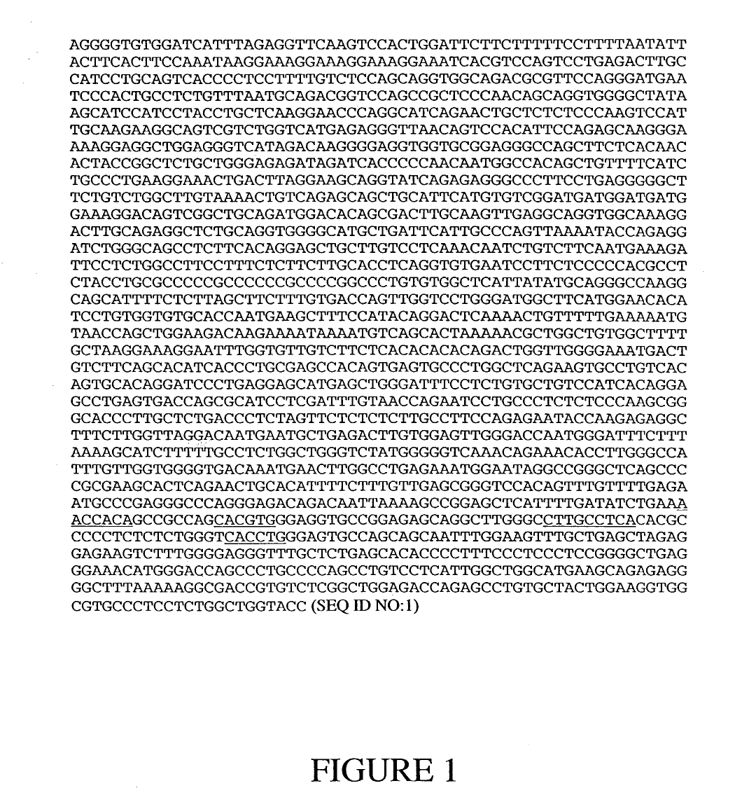

This invention also provides deletion constructs of the SOST regulatory region, as described in the Examples below, which either increase or decrease the regulatory activity beyond that of the full length 2003 nucleotide sequence. The deletion constructs are obtained by deleting from polynucleotide region of the invention a single nucleotide to a segment of many nucleotides (e.g., from 1 to 100 or more nucleotides) corresponding to the nucleotide sequence shown in FIG. 1 (SEQ ID NO: 1) to produce segments which have negative or positive regulatory activity to modify the rate of transcription from the regulatory polynucleotide. Deletion constructs in which negative regulatory regions have been removed result in enhanced transcription or expression activity. Such constructs provide greater sensitivity than the full-length regulatory region when used to screen for drugs which affect the regulatory activity of the polynucleotide regulatory region of the invention. Examples of these constructs are described below.

An isolated regulatory polynucleotide of the invention can be used as the regulatory region in a vector system such as an expression vector (see, e.g., Pouwels et al. Cloning Vectors: A Laboratory Manual, Elsevier, New York, (1985)). Such an expression vector containing a regulatory polynucleotide of the invention is useful in the production of recombinant polypeptides when a polynucleotide encoding the polypeptide is operable linked to a regulatory polynucleotide in the expression vector. General methods of expressing recombinant polypeptides are also known and are exemplified in R. Kaufman, Methods in Enzymology 185, 537-566 (1990). For example, an expression vector of the invention includes a regulatory polynucleotide of the invention 5′ (or upstream) of a cloning site (e.g., a multiple cloning site). Such cloning sites are known in the art and can be readily designed and ligated to a regulatory polynucleotide of the invention. In use a polynucleotide encoding a polypeptide to be expressed would be cloned, in frame, into the cloning site such that in the presence of a regulatory agent transcription of the polynucleotide would be induced or repressed by interaction of the regulatory polynucleotide and the regulatory agent. In one embodiment, a regulatory polynucleotide of the invention introduced into a recombinant host cell by transformation or transfection, for example, or by any other suitable method.

Established methods for introducing nucleic acid molecules (e.g., DNA and/or RNA) into mammalian cells have been described (Kaufman, Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). Electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). In addition, viral vectors can be used to deliver DNA or RNA to a mammalian cell. Such viral vectors include, for example, retroviral vectors, adenoviral vectors and the like. Selection of stable transformants can be performed using methods known in the art such as, for example, resistance to cytotoxic drugs or expression of a reporter gene (e.g., a luciferase gene, or the like). Kaufman et al., Meth. in Enzymology 185:487-511, 1990, describes several selection schemes, such as dihydrofolate reductase (DHFR) resistance. A suitable strain for DHFR selection can be CHO strain DX-B11, which is deficient in DHFR (Urlaub et al., Proc. Natl. Acad. Sci. USA 77:4216-4220, 1980). A plasmid expressing the DHFR cDNA can be introduced into strain DX-B11, and only cells that contain the plasmid can grow in the appropriate selective media. Examples of selectable markers that can be incorporated into expression vectors include cDNAs conferring resistance to antibiotics, such as G418 and hygromycin B. Cells having the vector can be selected based on resistance to such compounds.

Alternatively, a regulatory polynucleotide of the invention can be used to modulate expression of an endogenous (e.g., existing gene) in a cell by homologous recombination, or “gene targeting” techniques. Such techniques employ the introduction of a regulatory polynucleotide of the invention in a particular predetermined site on the genome, to induce expression of an endogenous gene product. The location of integration into a host chromosome or genome can be determined by one of skill in the art, given the known location and sequence of the gene. In one embodiment, the invention contemplates the introduction of an exogenous regulatory polynucleotide of the invention adjacent to a desired gene, to produce increased amounts of the gene product. The practice of homologous recombination or gene targeting is explained by Chappel in U.S. Pat. No. 5,272,071 (see also Schimke, et al. “Amplification of Genes in Somatic Mammalian cells,” Methods in Enzymology 151:85 (1987), and by Capecchi, et al., “The New Mouse Genetics: Altering the Genome by Gene Targeting,” TIG 5:70 (1989)).

A number of cell types may act as suitable host cells for transfection or transformation with a regulatory polynucleotide of the invention. Mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) as described by McMahan et al. (EMBO J. 10: 2821, 1991), human kidney 293 cells, human epidermal A431 cells, human Colo205 cells, other transformed primate cell lines, normal diploid cells, cell strains derived from in vitro culture of primary tissue (such as cardiovascular tissue, cartilage tissue, renal tissue, pulmonary tissue, musculoskeletal tissue, and neurological tissue), primary explants, HL-60, U937, HaK or Jurkat cells. In addition, the regulatory polynucleotide may be transfected or transformed into lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous polypeptides. Potentially suitable bacterial strains include Escherichia coli, Bacillus subtilis, and Salmonella typhimurium. As used herein, a host cell capable of expressing a polynucleotide operably linked to a regulatory polynucleotide of the invention is “transformed.” Cell-free transcription and translation systems could also be employed (see, e.g., U.S. Pat. No. 6,207,378, which is incorporated herein by reference). A host cell that comprises an isolated regulatory polynucleotide of the invention, including a host cell that comprise a regulatory polynucleotide operably linked to a reporter gene, is a “recombinant host cell”.

Also provided by the invention are expression cassettes comprising, 5′ to 3′ in the direction of transcription, a regulatory polynucleotide, a heterologous polynucleotide segment (such as, for example, a reporter gene) operatively associated with the regulatory polynucleotide, and, optionally, transcriptional and translational termination regions such as a termination signal and a polyadenylation signal. The foregoing polynucleotide, segment, and regions should be capable of operating in a transformed cell. The 3′ termination region may be derived from the same gene as the transcriptional initiation region or from a different gene. The expression cassette may be provided in a DNA construct that also has at least one replication system.

A heterologous polynucleotide or heterologous polynucleotide segment includes a polynucleotide (or polynucleotide segment) which is used to transform a cell by genetic engineering techniques, and which may not occur naturally in the cell. Structural polynucleotides are those portions of a polynucleotide which encode a protein, polypeptide, or portion thereof, possibly including a ribosome binding site and/or a translational start codon, but lack a regulatory region (e.g., a promoter). The term can also refer to copies of a structural polynucleotide naturally found within an organism but artificially introduced. Structural polynucleotides may encode a protein or polypeptide not normally found in the cell type or organism into which the polynucleotide is introduced and may include a regulatory polynucleotide to which it is operationally associated. As used herein, the term heterologous polynucleotide also includes polynucleotides coding for non-protein products, such as ribozymes or anti-sense molecules (see, e.g., U.S. Pat. No. 4,801,540).

Antisense RNA and DNA molecules typically act to directly block the translation of mRNA by hybridizing to targeted mRNA and preventing polypeptide translation. Antisense molecules that are complementary to a regulatory region of gene can serve to inhibit transcription by forming a triple helical structures that prevent transcription of the target gene (e.g., an SOST gene). (See generally, Helene, Anticancer Drug Des. 6(6):569-584, 1991; Helene, et al., Ann. N.Y. Acad. Sci., 660, 27-36, 1992; and Maher, Bioassays 14(12):807-815, 1992). Antisense approaches involve the design of oligonucleotides (either DNA or RNA) comprising sequences that are complementary to SEQ ID NO:1, its complement, or a fragment thereof. In particular, the EcoRV fragment of the SOST regulatory region, nucleotides 1673 to 1814 of SEQ ID NO: 1, is an appropriate target area due to its high promoter activity, and to the presence of a CbaI binding site and E box binding site, which enhance promoter activity, as described in the Examples below.

Effective antisense blocking of gene expression does not require absolute complementarity, although it is preferred. An oligonucleotide “complementary” to a portion of a nucleic acid molecule is a sequence having sufficient complementarity to be able to hybridize with the nucleic acid molecule, forming a stable duplex (or triplex, as appropriate). In the case of double-stranded antisense nucleic acid molecules, a single strand of the duplex DNA may thus be tested, or triplex formation may be assayed. The ability to hybridize will depend on both the degree of complementarity and the length of the antisense nucleic acid. Antisense nucleic acid molecules should be at least six nucleotides in length, but typically range from 6 to about 50 nucleotides in length. In one embodiment, an antisense molecule is at least 10, at least 17, at least 25, or at least 50 nucleotides in length. The antisense molecule can be DNA or RNA or chimeric mixtures or derivatives or modified versions thereof, single-stranded or double-stranded. The antisense molecule can be modified at the base moiety, sugar moiety, or phosphate backbone, for example, to improve stability of the molecule, hybridization, and the like. The antisense molecule may include other appended groups such as peptides (e.g., for targeting host cell receptors in vivo), or agents facilitating transport across the cell membrane (see, e.g., Letsinger et al., Proc. Natl. Acad. Sci. U.S.A. 86:6553-6556, 1989; Lemaitre et al., Proc. Natl. Acad. Sci. 84:648-652, 1987; PCT Publication No. WO88/09810), or hybridization-triggered cleavage agents or intercalating agents. (See, e.g., Zon, Pharm. Res. 5:539-549, 1988). The antisense molecules are delivered to cells that contain a regulatory polynucleotide of the invention. A number of methods have been developed for delivering antisense DNA or RNA to cells; e.g., antisense molecules can be injected directly into the tissue or cell or modified antisense molecules, designed to target the desired cells (e.g., antisense linked to peptides or antibodies that specifically bind receptors or antigens expressed on the target cell surface) can be administered systemically to a subject. One approach utilizes a recombinant DNA construct in which the antisense molecule is placed under the control of a strong pol III or pol II promoter. The use of such a construct to transfect target cells in the subject will result in the transcription of sufficient amounts of single stranded RNAs that will form complementary base pairs with the endogenous regulatory polynucleotide of the invention thereby forming a triple helix and preventing transcription. For example, a vector can be introduced in vivo such that it is taken up by a cell and directs the transcription of an antisense molecule. Such a vector can remain episomal or become chromosomally integrated so long as it can be transcribed to produce the desired antisense molecule. Such vectors can be constructed by recombinant DNA technology methods standard in the art. Vectors can be plasmid, viral, or others known in the art, used for replication and expression in mammalian cells.

Antisense molecules and triple helix molecules of the invention may be prepared by any method known in the art for the synthesis of DNA and RNA molecules. These include techniques for chemically synthesizing DNA or RNA oligonucleotides such as, for example, solid phase phosphoramidite chemical synthesis. Oligonucleotides can be synthesized by standard methods known in the art, e.g. by use of an automated DNA synthesizer (such as are commercially available from Biosearch and Applied Biosystems). As examples, phosphorothioate oligonucleotides may be synthesized by the method of Stein et al., Nucl. Acids Res. 16:3209, 1988. Methylphosphonate oligonucleotides can be prepared by use of controlled pore glass polymer supports (Sarin et al., Proc. Natl. Acad. Sci. U.S.A. 85:7448, 1988). Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense molecule constitutively or inducibly, depending on the promoter used, can be introduced stably into cell lines.

The invention also provides methods of screening for agents that modulate the regulatory activity of the regulatory polynucleotides of the invention, either by affecting signal transduction pathways that necessarily precede transcription or by affecting the regulatory polynucleotides directly.

For screening purposes an appropriate host cell such as, for example, a renal cell, a hepatocyte, a chondrocyte, an osteoclast, or an osteoblast, is transformed with an expression vector comprising a reporter gene operably linked to a regulatory polynucleotide of the invention. The transformed host cell is exposed to various test agents and then analyzed for expression of the reporter gene. This expression can be compared to expression from cells that were not exposed to the test agent. An agent that increases the activity of the regulatory polynucleotide will result in increased reporter gene expression relative to the control. Similarly, agents that act as antagonists for the regulatory polynucleotide pathway will result in decreased reporter gene expression relative to the control.

Thus one can screen for test agents that regulate the activity of the regulatory polynucleotide by:

(a) contacting a host cell containing a regulatory polynucleotide operably linked to a reporter gene with a test agent under conditions which allow for expression of the reporter gene;

(b) measuring the expression of the reporter gene in the presence of the test agent;

(c) measuring the expression of reporter gene in a control; and

(d) comparing a difference in expression between (b) and (c) to determine the ability of the test agent to regulate the activity of the regulatory polynucleotide.

Alternatively, a transformed cell may be induced with a transcriptional inducer, such as IL-1 or TNFα, forskolin, dibutyryl-cAMP, or a phorbol-type tumor promoter, such as, for example PMA. Transcriptional activity is measured in the presence or absence of a pharmacologic agent of known activity (e.g., a standard agent) or putative activity (e.g., a test agent). A change in the level of expression of a reporter gene in the presence of a test agent is compared to that effected by a standard agent. In this way, the ability of a test agent to affect transcription from a regulatory polynucleotide of the invention can be determined.

Thus, in another embodiment, the invention provides methods of measuring the ability of a test agent to modulate transcription from a regulatory polynucleotide of the invention by:

(a) contacting a host cell containing a regulatory polynucleotide operably linked to a reporter gene with an inducer of transcription from the regulatory polynucleotide under conditions which allow for expression of the reporter gene;

(b) measuring the expression of the reporter gene in the absence of the test agent;

(c) exposing the host cells to a test agent either prior to, simultaneous with, or after contacting, the host cells with the inducer;

(d) measuring the expression of the reporter gene in the presence of the test agent; and

(e) relating the difference in expression between (b) and (d) to the ability of the test agent to modulate transcription from the regulatory polynucleotide.

Since different inducers are known to affect different modes of signal transduction (e.g., cAMP responsive, calcium ion responsive), it is possible to identify with greater specificity agent that affect a particular signal transduction pathway that modulates transcription from the regulatory polynucleotide of the invention. Since the SOST gene product has been shown to be associated with bone formation (e.g., by suppressing bone formation; see e.g., Balemans et al., Human Mol. Genet. 10(5):537-543, 2001) such assays provide a means of identifying agents that will inhibit and/or promote bone formation by modulating transcription from the regulatory polynucleotide of the invention and thereby modulating SOST production. For example, by inhibiting SOST transcription the inhibitory effect of SOST activity on bone formation would be removed thereby promoting bone formation. Such agents would prove useful in treating bone degenerative disorder including, for example, osteoporosis. Other diseases, disorder, or defects that can be treated by the invention include, but are not limited to, non-union fractures; bone cavities; tumor resection; fresh fractures; cranial/facial abnormalities; spinal fusions, as well as those resulting from diseases such as cancer, arthritis, including osteoarthritis, and bone cysplasia sclerosteosis. In addition, the compositions, methods and agents identified by the methods of the invention can be used for treating such diseases and disorders as those selected from the group consisting of fracture repair, promoting or inhibiting bone in-growth into a prosthesis, promoting union of an area of non-union, promote healing of non-healing wounds, and promoting the integration of dental implants into bone.

The regulatory polynucleotides of the present invention are useful in effecting tissue specific expression in various cell types including, for example, osteoblasts, osteoclasts, hepatocytes, chondrocytes, and renals cells and screening for drugs that selectively modulate gene expression in such cells and drugs that modulate bone synthesis and resorption processes.

The terms “treat”, “treating”, and “treatment” used herein include curative, preventative (e.g., prophylactic) and palliative or ameliorative treatment. For such therapeutic uses, an agent identified by the method of the invention, constructs containing a regulatory polynucleotide of the invention, and expression cassettes of the invention can be administered to the subject through known methods of administration.

In practicing a method of treatment or use of the invention, a therapeutically effective amount of a therapeutic agent of the invention is administered to a subject having a condition to be treated, typically to treat or ameliorate diseases associated with abnormal bone formation, including, for example, osteoporosis; non-union fractures; bone cavities; tumor resection; fresh fractures; cranial/facial abnormalities; spinal fusions; as well as those resulting from diseases such as cancer; arthritis, including osteoarthritis; bone cysplasia sclerosteosis; promoting or inhibiting bone in-growth into a prosthesis; promote healing of non-healing wounds; and promoting the integration of dental implants into bone. “Therapeutic agent” includes, without limitation, a regulatory polynucleotide of the present invention thereof; as well as agents that modulate the activity of a regulatory polynucleotide of the invention which agent includes those identified by the methods of the invention. As used herein, the term “therapeutically effective amount” means the total amount of each therapeutic agent or other active component of the pharmaceutical composition or method that is sufficient to show a meaningful subject benefit, e.g., treatment, healing, prevention or amelioration of a relevant medical condition, or an increase in rate of treatment, healing, prevention or amelioration of such conditions. When applied to an individual therapeutic agent or active ingredient, administered alone, the term refers to that ingredient alone. When applied to a combination, the term refers to combined amounts of the ingredients that result in the therapeutic effect, whether administered in combination, serially or simultaneously. As used herein, the phrase “administering a therapeutically effective amount” of a therapeutic agent means that the subject is treated with the therapeutic agent in an amount and for a time sufficient to induce an improvement in at least one indicator that reflects the severity of the disorder. An improvement is considered “sustained” if the subject exhibits the improvement on at least two occasions separated by one or more weeks. The degree of improvement is determined based on signs or symptoms, and determinations may also employ questionnaires that are administered to a human subject, such as quality-of-life questionnaires. Various indicators that reflect the extent of the subject's illness may be assessed for determining whether the amount and time of the treatment is sufficient. The baseline value for the chosen indicator or indicators is established by examination of the subject prior to administration of the first dose of the therapeutic agent. Typically, the baseline examination is done within about 60 days of administering the first dose. If the therapeutic agent is being administered to treat acute symptoms, the first dose is administered as soon as practically possible after the injury has occurred. Improvement is induced by administering therapeutic agents until the subject manifests an improvement over baseline for the chosen indicator or indicators (e.g., bone mass and/or strength). In treating chronic conditions, this degree of improvement is obtained by repeatedly administering this therapeutic composition over a period of at least a month or more, e.g., for one, two, or three months or longer, or indefinitely. A period of one to six weeks, or even a single dose, often is sufficient for treating acute conditions. For injuries or acute conditions, a single dose may be sufficient. Although the extent of the subject's illness after treatment may appear improved according to one or more indicators, treatment may be continued indefinitely at the same level or at a reduced dose or frequency. Once treatment has been reduced or discontinued, it later may be resumed at the original level if symptoms should reappear.

One skilled in the pertinent art will recognize that suitable dosages will vary, depending upon such factors as the nature and severity of the disorder to be treated, the subject's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the agent and can be readily determined by routine experimentation. A dose may be formulated in animal models to achieve a circulating plasma concentration range that includes the IC₅₀ (i.e., the concentration of test agent that achieves a half-maximal inhibition of symptoms) as determined in cell culture, while minimizing toxicities. Such information can be used to more accurately determine useful doses in humans. Ultimately, the attending physician will decide the amount of a therapeutic agent or diagnostic agent to treat each individual subject.

Compositions comprising an effective amount of a therapeutic agent of the invention will typically be in combination with other components such as a physiologically acceptable diluent, carrier, or excipient, are provided herein. The term “pharmaceutically acceptable” means a non-toxic material that does not interfere with the effectiveness of the biological activity of the active ingredient(s). Formulations suitable for administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The therapeutic agent can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, and the like, or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations are within the level of skill in the art, as disclosed, for example, in U.S. Pat. Nos. 4,235,871; 4,501,728; 4,837,028; and 4,737,323. Such compositions will influence the physical state, solubility, stability, rate of in vivo release, or rate of in vivo clearance, and are thus chosen according to the intended application, so that the characteristics of the carrier will depend on the selected route of administration. In one embodiment of the invention, sustained-release forms of a therapeutic agent are used. Sustained-release forms suitable for use in the disclosed methods include, but are not limited to, therapeutic agents of the invention encapsulated in a slowly-dissolving biocompatible polymer (such as the alginate microparticles described in U.S. No. 6,036,978), admixed with such a polymer (including topically applied hydrogels), and or encased in a biocompatible semi-permeable implant.

The pharmaceutical composition may further contain other agents that either enhance the activity of the agent or polynucleotide or compliment its activity or use in treatment (e.g., other bone modulating agents such as bisphosphonate, and the like). Such additional factors and/or agents may be included in the pharmaceutical composition to produce a synergistic effect, or to minimize side effects. Examples of drugs to be administered concurrently include, but are not limited to, antivirals, antibiotics, analgesics, corticosteroids, antagonists of inflammatory cytokines, non-steroidal anti-inflammatories, pentoxifylline, thalidomide, and disease-modifying antirheumatic drugs (DMARDs) such as azathioprine, cyclophosphamide, cyclosporine, hydroxychloroquine sulfate, methotrexate, leflunomide, minocycline, penicillamine, sulfasalazine and gold compounds such as oral gold, gold sodium thiomalate, and aurothioglucose.

Any efficacious route of administration may be used to administer a therapeutic or diagnostic agent of the invention. Parenteral administration includes injection, for example, via intra-articular, intravenous, intramuscular, intralesional, intraperitoneal, or subcutaneous routes by bolus injection or by continuous infusion, and also includes localized administration, e.g., at a site of disease or injury. Other suitable means of administration include sustained release from implants (e.g., matrices, sponges, pins and the like to repair bone); aerosol inhalation and/or insulation; eyedrops; vaginal or rectal suppositories; buccal preparations; oral preparations, including pills, syrups, lozenges or chewing gum; and topical preparations such as lotions, gels, sprays, ointments or other suitable techniques. Alternatively, a nucleic acid construct comprising (1) a regulatory polynucleotide of the invention operable linked to a reporter gene or therapeutic gene, or (2) an antisense (e.g., triplex forming) molecule may be administered by implanting recombinant or host cells that express the construct or antisense molecule. The cells may be engineered ex vivo or the construct delivered in vivo to produce recombinant cells. A construct or antisense molecule can be introduced into a subject's cells, for example, by injecting naked DNA or liposome-encapsulated DNA containing a regulatory polynucleotide of the invention or an antisense molecule of the invention, or by other means of transfection. Polynucleotides of the invention may also be administered to subjects by other known methods for introduction of nucleic acids into a cell or organism (including, without limitation, in the form of viral vectors).

When a therapeutic agent of the invention is administered orally, the agent will typically be in the form of a tablet, capsule, powder, solution or elixir. When administered in tablet form, the pharmaceutical composition of the invention may additionally contain a solid carrier such as a gelatin or an adjuvant. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol.

When a therapeutic agent of the invention is administered by intravenous, cutaneous or subcutaneous injection, the agent will typically be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, cutaneous, or subcutaneous injection should contain, in addition to the therapeutic agent of the invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the invention may also contain stabilizers, preservatives, buffers, antioxidants, or other additives known to those of skill in the art. The duration of intravenous therapy using the pharmaceutical composition of the invention will vary, depending on the severity of the disease being treated and the condition and potential idiosyncratic response of each individual subject. Ultimately the attending physician will decide on the appropriate duration of intravenous therapy using the pharmaceutical composition of the invention.

For compositions of the invention which are useful in treating bone, cartilage, tendon or ligament disorders, the therapeutic method includes administering the composition topically, systematically, or locally as an implant or device. When administered, the therapeutic composition for use in the invention is in a pyrogen-free, physiologically acceptable form. Further, the composition may desirably be encapsulated or injected in a viscous form for delivery to the site of bone, cartilage, or tissue damage. Topical administration may be suitable for wound healing and tissue repair.

In addition to human subjects, the therapeutic agents are useful in the treatment of disease conditions in non-human animals, such as pets (canine, feline, avian, primates species, and the like), domestic farm animals (equine, bovine, porcine, avian species, and the like). In such instances, an appropriate dose may be determined according to the animal's body weight. In one embodiment, the regulatory polynucleotide is constructed from genes derived from the same species as the subject.

In another aspect of this invention, transgenic animals expressing a heterologous polynucleotide encoding a detectable product under the regulatory control of a regulatory polynucleotide of the invention may be used to determine the effect of a test agent on the stimulation or inhibition of the regulatory polynucleotide. The test agent is administered to the animal and the degree of expression of the heterologous polynucleotide observed is compared to the degree of expression in the absence of administration of the test agent using routine bioassays as disclosed herein. Such transgenic animals can prove useful as disease models for studying SOST function, bone formation, and the like.

Transgenic animals with genes comprising a regulatory polynucleotide of the invention operably linked to a heterologous gene can be prepared by methods known to those of skill in the art such as, but not limited to, B. Hogan et al., Manipulating the Mouse Embryo, Cold Spring Harbor Laboratory Press, New York (1986) and U.S. Pat. No. 5,162,215.

For example, using mice, fertilized eggs are collected by washing out the oviducts of mated females and an expression cassette comprising the regulatory polynucleotide operably linked to a heterologous polynucleotide is microinjected into the pronuclei. The injected eggs are then transferred to and implanted in the uterus of foster mothers, female mice made pseudopregnant by mating with vasectomized males. After birth the progeny mice are checked for presence of the transgene by Southern blotting of DNA extracted from a small piece of the tail. If suitable primers are available, screening can be rapidly performed by polymerase chain reaction (PCR). The transgene may be integrated into the germ line cell, somatic cells or both. Transgenic mice carrying the transgene in their germ line cells can be identified by mating them with normal nontransgenic mice and determining whether the inheritance of the transgene follows expected Mendelian genetics. This is often conveniently accomplished by including in the injected expression cassette a gene coding for readily visible trait such as skin coat color. An alternative method of transgenic animal production involves injecting an expression cassette comprising the regulatory polynucleotide of the invention into undifferentiated embryonic stem cells prior to injecting into the mouse blastocyst.

The transgenic animals of the invention are useful as models for studying the function of the SOST gene, for studying the etiology of various bone degenerative diseases or disorders including for example, osteoporosis, bone dysplasia sclerosteosis, and the like, and for studying the activity of various drugs and drug candidates in treating such diseases and disorder.

Unless otherwise defined, all technical and scientific terms used herein have the same meaning as commonly understood by one of ordinary skill in the art to which this invention belongs. All headings and subheading provided herein are solely for ease of reading and should not be construed to limit the invention. The terms “a”, “an” and “the” as used herein are meant to encompass the plural unless the context clearly dictates the singular form. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. The following examples are intended to illustrate particular embodiments and not to limit the scope of the invention.

### EXAMPLE 1

### Identifying the SOST Regulatory Region

The polynucleotide regulatory region for the SOST gene was identified by comparing human and mouse 5′ untranslated regions (UTRs). The 5′UTR of the human SOST gene having accession no. AF326736 (the content of which is incorporated herein) was aligned and compared to the 5′UTR of the mouse SOST gene having accession no. AF326737 (the content of which is incorporated herein). Based upon the alignment a conserved sequence of approximately 2 kb was identified and is presented in SEQ ID NO:1. The region selected for further study was isolated from human genomic DNA (Promega) by PCR using the following 5′ and 3′ primers respectively:

(SEQ ID NO:6)

5′-TCTCCCCCGGGTGTGGATCATTTAGAGGTTCAAG-3′
and

(SEQ ID NO:7)

5′-GCCCTAGATCTCCCAAAGACTTCTCCTCTAGCTC-3′

PCR was carried out using HotStarTaq master mix (Qiagen) with the following conditions: 95° C. for 15 minutes, followed by 30 cycles of 95° C. for 1 min., 55° C. for 1 min., 72° C. for 3 min.

### EXAMPLE 2

### Function of Regulatory Region in Two Cell Lines

Two human osteosarcoma cell lines, SAOS cells, and MG63 cells (both obtained from ATCC) were used to test the ability of the polynucleotide regulatory region of SEQ ID NO:1 to promote transcription intracellularly. SAOS cells are considered to be a more differentiated osteoblast cell type compared to MG-63 cells in terms of appearance and enhanced expression of alkaline phosphatase.

Both cell lines were first tested for SOST gene expression using quantitative real-time Taqman PCR relative to the HPRT (hypoxanthine phosphoribosyltransferase) housekeeper gene. Basal SOST gene expression was detected in SAOS cells, but not in MG-63 cells. Additionally, SOST expression was shown to be upregulated by the addition of Vitamin D to SAOS culture media to be weakly induced by a combination of Vitamin D and Osteogenesis Induction Medium (OIM) for MG-63 cells. Prior to RNA harvest, cells were treated for 72 hours with Vitamin D₃, OIM, Vitamin D₃ plus OIM or no treatment. To test whether the SOST regulatory region promotes transcription in these two cell lines, the PCR product (SEQ ID NO: 1) described above was digested with SmaI and BglII restriction enzymes to produce polynucleotides 1 to 1814 of SEQ ID NO: 1, which was inserted into a promoterless pGL2-Basic luciferase reporter vector (Promega, Madison, Wis.) digested with the same enzymes (hereinafter referred to as the SOST-luc reporter). The region inserted corresponds approximately to nucleotides −2000 to −190 of the SOST gene locus relative to the position of +1 of the initiation methionine for the SOST open reading frame. The expression of SOST-luc was compared with a promoterless pGL2-Basic luciferase activity without the SOST regulatory region (pGL2-Basic). In addition, a pRL-TK plasmid containing a Herpes Simplex Virus Thymidine Kinase (HSV-TK) promoter driving expression of Secreted Alkaline Phosphatase (SEAP, Clontech) was co-transfected and luciferase data was normalized to SEAP activity to determine transfection efficiency. SOAS and MG-63 cells were transiently transfected with 1 ug of SOST-luc or pGL2-Basic and analyzed 48 hours after transfection. For transfection, cells were plated at a density of 2.5×10⁶ cells/well in 6-well tissue culture plates (Corning, N.Y.). After 24 hr, cells were transfected in duplicate with a mixture of FuGene 6 Transfection Reagent (Roche), MEM, and 1 ug DNA/well, including 20 ng/well of the pSEAP2-control secreted alkaline phosphatase vector (Clontech, Palo Alto, Calif.). p-Bluescript (Stratagene) was used when needed to provide a final total of 1 ug DNA per well. 48 hrs after transfection, cell supernatants (100 ul) were harvested and assayed using a SEAP Chemiluminescence Detection Kit (Clontech). Cell lysates were harvested using the Bright-Glo Luciferase Assays System (Promega, Madison, Wis.). SEAP and luciferase samples were transferred to white opaque 96-well plates (Costar) and assayed using a MicroBeta Trilux luminescence counter (Wallac, Finland). Luciferase values were normalized to SEAP data to correct for well-to-well variations in transfection efficiency. Each construct shown was tested in a minimum of two experiments with consistent results.

The SOST-luc reporter consistantly showed a 5-fold increase in luciferase activity compared to the control vector in SAOS cells. No increase in luciferase activity over the control was detected in MG-63 cells. This was consistent with the preliminary determination that SAOS cells express SOST mRNA and MG-63 cells do not express SOST mRNA.

### EXAMPLE 3

### Deletion Analysis of SOST Regulatory Region

To analyze the regulatory sequences required for SAOS-specific SOST expression, a series of deletion constructs was generated from the SOST regulatory region in a 5′ to 3′ direction by digesting the native restriction sites with the following restriction enzymes MluI, HpaI, SphI, HindIII, BamHI, and EcoRV. The restriction sites in the SOST regulatory region are shown in FIG. 2A. After digestion with the restriction enzyme, the fragments are treated with Klenow or T4 DNA polymerase to fill in overhangs, digested with SmaI and BglII and inserted into pGL2-Basic also digested with SmaI and BglII. Smaller deletion and point mutants were generated between the EcoRV and BglII sites was using PCR-based approach with oligonucleotides spanning the ends of the desired sequence and containing BglII, EcoRV or SmaI restriction sites. Point mutations were generated using site-directed mutagenesis. SAOS cells were transfected as described above with the various fragment constructs as well as the control vector.

As can be seen in FIG. 2, deletion of 5′ regions encompassing the well-conserved regions (the portion of the regulatory region of highest homology between species, shown in FIG. 2) did not decrease luciferase activity but instead increased activity by approximately two to three fold. The greatest promoter activity was found for the EcoRV to BglII fragment alone. This fragment is about 142 nucleotides in length, and corresponds to nucleotides 1673 to 1814 of SEQ ID NO: 1, which is −331 to −190 nucleotides relative to the initiation methionine of the SOST coding region. As is seen in FIG. 2, the increase in luciferase activity is almost three fold over the 1.8 kb SOST regulatory region. This indicates a repressive effect exerted by the 5′ sequences of the 1.8 kb region, as seen in FIG. 2.

Luciferase assays using finer scale deletion constructs of the EcoRV to BglII fragments were performed a described above, and indicated that the 5′ region of the EcoRV fragment contained important regulatory elements. Analysis of the EcoRV to BglII fragment sequence identified consensus binding sites for transcription factors. These are, in a 5′ to 3′ direction, a Cbfa1 binding site, AACCACA (SEQ ID NO: 2), an upstream E box, CACGTG (SEQ ID NO: 3), a C/EBP binding site, CTTGCCTCA (SEQ ID NO: 4), and a downstream E box, CACCTG (SEQ ID NO: 5). These sites are underlined in FIG. 1.

Cbfa1/Runx2, referred to as Cbfa1, is a transcription factor shown to be essential for oteoblast differentitation and bond formation during embryogenesis (Banerjee et al., PNAS 93:4968 (1996); Ducy et al. Mol. Cell Biol. 15:1858 (1995)). The C/EBP (CCAAT/enhancer binding proteins) is a family of transcription factors associated with differentiation of a number of tissues. See, for example, Piontkewitz et al. Dev Biol 179:288(1996), Descombes et al. Cell 67:569 (1991), Sterneck et al. Genes Dev 11:2153 (1997). The E box is a binding site for Myc/Max family of proteins (Dang et al. PNAS 89:599 (1992)).

### EXAMPLE 4

### Identification of Factors Enhancing Transcription

Additional studies were conducted to determine if any of the above transcription factors having binding sites in the EcoRV to BglII fragment of the SOST regulatory region were responsible for the difference in SOST-luc expression in SAOS compared with MG-63 cells.

Deletion analysis of the EcoRV to BglII fragment of the SOST regulatory region indicated that the 5′ 75 nucleotides of this fragment retained about 90% of the functional activity of this fragment. This 75 nt fragment was then used as a probe for gel mobility shift assays in order to identify the protein or proteins which bound in the SOST-expressing SAOS cells. Nuclear extracts were prepared from untreated SAOS and MG-63 cells and incubated with the ³²P-labeled 75 nucleotide as a probe, followed by gel electrophoresis. Both SAOS and MG-63 nuclear extracts produced a slow-migrating band of essentially equal mobility, but SAOS extracts also produced a faster-migrating band that was undetected in MG-63 cells. To identify the region of the probe responsible for the SAOS-specific band, the 75 nucleotide probe was divided into A, B, C, and D subfragments, wherein A contained the Cbfa1 binding site, B contained the upstream E box, C contained no known binding sites, and D contained the downstream E box. ³²P-labeled labelled subfragments were annealed with SAOS And MG-63 nuclear extracts. Probes B and D weakly bound to factors present in both MG-63 and SAOS cells, whereas probe A strongly and specifically bound to a factor present only in SAOS cells. Probe A contained the Cbfa1 binding site.

This finding was confirmed using unlabeled annealed oligonucleotides representing regions A to D as competitors to the 1.8 kb SOST regulatory region probe in the presence of SAOS nuclear extracts. The unlabeled 1.8 kb probe was an effective competitor for both shifted bands at all concentrations used. Unlabeled probe A efficiently competed with the SAOS-specific band but not the shared band, whereas probes B and D eliminated the band present in both cell lines. A version of probe B bearing a mutated E box consensus site was an ineffective cold competitor, indicating that specific binding to E box sequences was required for successful competition by the sequences in region B.

Mutation-bearing oligonucleotides were utilized in further competition experiments similar to those described above to define a 9-nucleotide region including the Cbfa1 consensus site as the SAOS-specific regulatory element. To confirm the specificity of Cbfa1 binding to its binding site in the SOST regulatory region, a series of competitor probes were constructed bearing individual mutations in each of these 9 nucleotides as well as oligonucleotides in which all nucleotides outside the 9-nucleotide core were changed. Each mutant oligonucleotide pair was annealed and tested for its ability to block binding between the SAOS nuclear extract and the ³²P-labelled A probe. The results indicated that only the central 9 nucleotides are necessary for binding to the SAOS-specific element. The most effective mutations, and therefore the poorest competitors, were those which changed the two central cytosine nucleotides to adenine, consistent with the requirement for cytosine residues at these positions in the Cbfa1 consensus. Mutation of adenine to guanine generally had little effect, a result attributable to the ability of Cbfa1 to recognize either A or G at these positions. Overall, the results were in good agreement with the identification of the SAOS-specific band in these assays as Cbfa1.

To further confirm that Cbfa1 participated in the binding of SAOS nuclear extracts to probe A, a gel supershift analysis was performed using antibodies directed against either Cbfa1 or a control protein (integrin β2) (goat polyclonal antibodies, Santa Cruz Biotechnology). Addition of the Cbfa1 antibody to a binding reaction containing SAOS nuclear extract and probe A resulted in a supershifted band of decreased mobility, whereas incubation with the control antibody had no effect. This result confirmed that Cbfa1 is present in the SAOS-specific complex that binds to probe A.

If Cbfa1 is a regulator of SOST expression, then differential expression of Cbfa1 between SAOS and MG-63 cells might account for the specific expression of SOST in SAOS cells. Taqman analysis was used to quantitatively compare Cbfa1 expression between the two cell lines. Cbfa1 expression was undetectable in untreated MG-63 cells, but was robust in SAOS cells under all conditions tested, consistent with a role for Cbfa1 in SOST promoter regulation in SAOS cells. In both cell lines, Cbfa1 could be upregulated by treatment with a combination of Vitamin D3 and osteogenesis induction medium (OIM), but Cbfa1 expression remained higher in SAOS cells under all conditions tested.

To determine whether transfected Cbfa1 could drive further increases in the transcriptional activity of the SOST promoter, the Cbfa1 gene was recovered from an SAOS cDNA library and inserted it into a mammalian expression vector. When overexpressed in SAOS cells, Cbfa1 increased activity of both the EcoRV fragment and the 1.8 kb SOST regulatory region, further confirming that SOST is a Cbfa1 target gene.

Deletion and site-directed mutagenesis experiments showed that while the downstream E box sequence is dispensable for SOST promoter activity in SAOS cells, the upstream E box appears to be functional. Deletion or point mutation of this E box sequence resulted in a consistent 3-fold decrease in SOST promoter activity. Additionally, as previously described, oligonucleotides bearing a mutated E box were unable to compete for binding of the slower-mobility band found in both SAOS and MG-63 nuclear extracts, whereas the wildtype E box made an effective competitor.

To ascertain whether any members of the MyoD family might be expressed in SAOS and/or MG-63 cells and could therefore transactivate the SOST regulatory region, semi-quantitative RT-PCR analysis was performed on both cell types. MyoD was expressed at similar levels in both SAOS and MG-63 cells, consistent with the observation that the MyoD binding site was bound by factors present in both SAOS and MG-63 nuclear extracts.

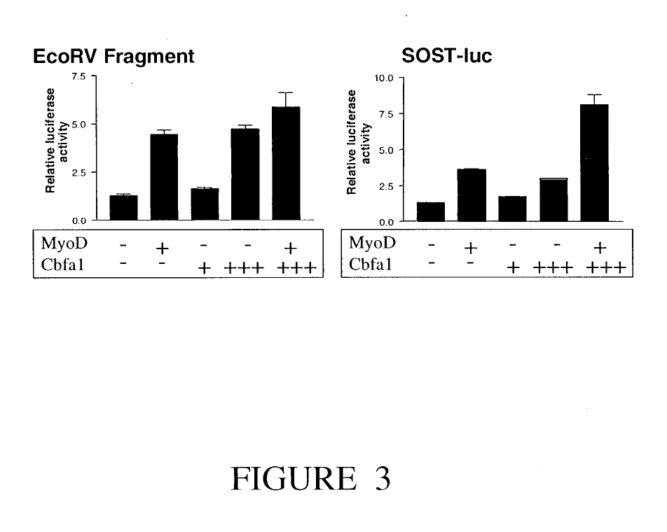

To test the effects of the two proteins Cbaf1 and MyoD on transactivitation of the 1.8 kb SOST regulatory region, the following tests were performed. SAOS cells were transfected with 500 ng of either the EcoRV fragment (nucleotides 1673 to 1814 of SEQ ID NO: 1) or 1.8 kb SOST regulatory region (nucleotides 1 to 1841 of SEQ ID NO:1) and the indicated combinations of expression plasmids for MyoD (150 ng) or Cbfa1 (30 ng, 300 ng). pBluescript (Stratagene) was used as needed to bring the DNA total to 1 μg per well, and SEAP (20 ng) was used to normalize for transfection efficiency. Cells were harvested 48 hrs after transfection. The results are shown in FIG. 3. Both the SOST-luc and the EcoRV fragment were activated by Cbaf1 and MyoD.

These results demonstrate that the SOST regulatory region can be transactivated by both Cbfa1 and MyoD, through binding at the Cbfa1 binding site and the upstream E box. Thus portions of the EcoRV sequence would be particularly effective targets for inhibiting SOST expression using antisense molecules or other molecules.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An isolated nucleic acid molecule comprising a regulatory polynucleotide selected from the group consisting of:
(a) a regulatory polynucleotide having a nucleotide sequence at least 80% identical to SEQ ID NO:1 or its complement, and that regulates the expression of a nucleic acid molecule operably linked thereto;
(b) a regulatory polynucleotide that hybridizes to a polynucleotide having the sequence set forth in SEQ ID NO:1 or its complement, under moderate to high stringency conditions, and that regulates the expression of a nucleic acid molecule operably linked thereto; and
(c) a regulatory polynucleotide comprising a fragment of (a) or (b), or a polynucleotide having at least 80% sequence identity to a fragment of (a) or (b), and that regulates the expression of a nucleic acid molecule operably linked thereto.

2. The isolated nucleic acid molecule of claim 1, wherein the regulatory polynucleotide consists essentially of nucleotides 1 to 1814 of SEQ ID NO: 1, its complement, or a fragment thereof, and that regulates the expression of a nucleic acid molecule operably linked thereto.

3. The isolated nucleic acid molecule of claim 1, wherein the regulatory polynucleotide has a sequence at least 80% identitical to nucleotides 1 to 1814 of SEQ ID NO: 1, or its complement, or a fragment thereof, and that regulates the expression of a nucleic acid molecule operably linked thereto.

4. The nucleic acid molecule of claim 1, wherein the regulatory polynucleotide consists essentially of nucleotides 1673 to 1814 of SEQ ID NO:1, its complement, or a fragment thereof, and that regulates the expression of a nucleic acid molecule operably linked thereto.

5. The isolated nucleic acid molecule of claim 1, wherein the regulatory polynucleotide has a sequence at least 80% identical to nucleotides to 1673 to 1814 of SEQ ID NO: 1, its complement, or a fragment thereof, and that regulates the expression of a nucleic acid molecule operably linked thereto

6. An expression vector comprising the nucleic acid molecule of claim 1.

7. A recombinant host cell genetically engineered to contain a nucleic acid molecule of claim 1.

8. A host cell containing the expression vector of claim 6.

9. The host cell of claim 8, wherein the host cell is an osteoclast, an osteoblast, a chondrocyte, a hepatocyte, or a renal cell.

10. A composition comprising the expression vector of claim 6 and a pharmaceutically acceptable carrier.

11. A composition comprising a recombinant host cell of claim 7 and a pharmaceutically acceptable carrier.

12. A method for identifying an agent that alters transcription comprising contacting a sample containing a regulatory polynucleotide operably linked to a reporter gene with the agent and determining expression of the reporter gene compared to a control, wherein a change in expression is indicative that the agent alters transcription, wherein the regulatory polynucleotide is selected from the group consisting of:
(a) a regulatory polynucleotide having a nucleotide sequence at least 80% identical to SEQ ID NO:1 or the complement thereof, and that regulates the expression of a nucleic acid molecule operably linked thereto;
(b) a regulatory polynucleotide that hybridizes to a polynucleotide having the sequence set forth in SEQ ID NO:1 or its complement, under moderate to high stringency conditions, and that regulates the expression of a nucleic acid molecule operably linked thereto; and
(c) a regulatory polynucleotide comprising a fragment of (a) or (b), or a polynucleotide having at least 80% sequence identity to a fragment of (a) or (b), and that regulates the expression of a nucleic acid molecule operably linked thereto.

13. The method of claim 12, wherein the reporter gene is selected from the group consisting of a chloramphenicol acetyl transferase (CAT), a luciferase, a β-galactosidase, an alkaline phosphatase, an antibiotic resistance gene, an SV40 T antigen, sclerosteosis protein (SOST), and a human growth hormone (hGH).

14. The method of claim 12, wherein the agent is selected from the group consisting of a polynucleotide, a polypeptide, a peptide, a peptidomimetic, and a small molecule.

15. The method of claim 14, wherein the polynucleotide is an antisense molecule.

16. An agent identified by the method of claim 12.

17. A method of modulating bone formation in a subject, comprising administering the agent of claim 16.

18. The method of claim 17 wherein the agent is selected from the group consisting of a polynucleotide, a polypeptide, a peptide, a peptidomimetic, and a small molecule.

19. The method of claim 18, wherein the polynucleotide is an antisense molecule.

20. A method of treating a bone degenerative disease or disorder in a subject comprising administering an agent to the subject which interacts with a regulatory polynucleotide of claim 1 and inhibits expression of an SOST gene product 3′ of the regulatory polynucleotide.

21. The method of claim 20, wherein the bone degenerative disease or disorder is selected from the group consisting of non-union fractures; bone cavities; tumor resection; fresh fractures; cranial/facial abnormalities; spinal fusions; cancer; arthritis; osteoarthritis; and osteoporosis.

22. The method of claim 20, wherein the agent is selected from the group consisting of a polynucleotide, a polypeptide, a peptide, a peptidomimetic, and a small molecule.

23. The method of claim 22, wherein the polynucleotide is an antisense molecule.

24. The method of claim 20, further comprising administering an additional agent that stimulates bone formation.

25. The method of claim 24, wherein the additional agent is a bisphosphonate and/or parathyroid hormone.

26. A method of promoting bone formation in a subject comprising administering an agent to a subject which interacts with the regulatory polynucleotide of claim 1 and inhibits expression of an SOST gene product 3′ of the regulatory polynucleotide.


SEQUENCE LISTING (7 sequences)

SEQ ID NO: 1 - DNA, 2003 bp, Homo sapiens
aggggtgtggatcatttagaggttcaagtccactggattcttctttttccttttaatattacttcacttccaaataaggaaaggaaaggaaaggaaatcacgtccagtcctgagacttgccatcctgcagtcacccctccttttgtctccagcaggtggcagacgcgttccagggatgaatcccactgcctctgtttaatgcagacggtccagccgctcccaacagcaggtggggctataagcatccatcctacctgctcaaggaacccaggcatcagaactgctctctcccaagtccattgcaagaaggcagtcgtctggtcatgagagggttaacagtccacattccagagcaagggaaaaggaggctggagggtcatagacaaggggaggtggtgcggagggccagcttctcacaacactaccggctctgctgggagagatagatcacccccaacaatggccacagctgttttcatctgccctgaaggaaactgacttaggaagcaggtatcagagagggcccttcctgagggggcttctgtctggcttgtaaaactgtcagagcagctgcattcatgtgtcggatgatggatgatggaaaggacagtcggctgcagatggacacagcgacttgcaagttgaggcaggtggcaaaggacttgcagaggctctgcaggtggggcatgctgattcattgcccagttaaaataccagaggatctgggcagcctcttcacaggagctgcttgtcctcaaacaatctgtcttcaatgaaagattcctctggccttcctttctcttcttgcacctcaggtgtgaatccttctcccccacgcctctacctgcgcccccgccccccgccccggccctgtgtggctcattatatgcagggccaaggcagcattttctcttagcttctttgtgaccagttggtcctgggatggcttcatggaacacatcctgtggtgtgcaccaatgaagctttccatacaggactcaaaactgtttttgaaaaatgtaaccagctggaagacaagaaaataaaatgtcagcactaaaaacgctggctgtggcttttgctaaggaaaggaatttggtgttgtcttctcacacacacagactggttggggaaatgactgtcttcagcacatcaccctgcgagccacagtgagtgccctggctcagaagtgcctgtcacagtgcacaggatccctgaggagcatgagctgggatttcctctgtgctgtccatcacaggagcctgagtgaccagcgcatcctcgatttgtaaccagaatcctgccctctctcccaagcgggcacccttgctctgaccctctagttctctctcttgccttccagagaataccaagagaggctttcttggttaggacaatgaatgctgagacttgtggagttgggaccaatgggatttctttaaaagcatctttttgcctctggctgggtctatgggggtcaaacagaaacaccttgggccatttgttggtggggtgacaaatgaacttggcctgagaaatggaataggccgggctcagccccgcgaagcactcagaactgcacattttctttgttgagcgggtccacagtttgttttgagaatgcccgagggcccagggagacagacaattaaaagccggagctcattttgatatctgaaaaccacagccgccagcacgtgggaggtgccggagagcaggcttgggccttgcctcacacgccccctctctctgggtcacctgggagtgccagcagcaatttggaagtttgctgagctagaggagaagtctttggggagggtttgctctgagcacacccctttccctccctccggggctgagggaaacatgggaccagccctgccccagcctgtcctcattggctggcatgaagcagagaggggctttaaaaaggcgaccgtgtctcggctggagaccagagcctgtgctactggaaggtggcgtgccctcctctggctggtacc

SEQ ID NO: 2 - DNA, 7 bp, Homo sapiens
aaccaca

SEQ ID NO: 3 - DNA, 6 bp, Homo sapiens
cacgtg

SEQ ID NO: 4 - DNA, 9 bp, Homo sapiens
cttgcctca

SEQ ID NO: 5 - DNA, 6 bp, Homo sapiens
cacctg

SEQ ID NO: 6 - DNA, 34 bp, Artificial Sequence
  Primers
tctcccccgggtgtggatcatttagaggttcaag

SEQ ID NO: 7 - DNA, 34 bp, Artificial Sequence
  Primers
gccctagatctcccaaagacttctcctctagctc